(19) United States
(12) Patent Application Publication
(10) Pub. No.: US 2020/0069854 A1
(43) Pub. Date: Mar. 5, 2020

(54) TOTAL HEART ASSISTANCE DEVICE

(71) Applicant: SAMUEL GUELLER, Cincinnati, OH (US)

(72) Inventor: SAMUEL GUELLER, Cincinnati, OH (US)

(21) Appl. No.: 16/122,815

(22) Filed: Sep. 5, 2018

(51) Int. Cl. A61M 1/10

(52) U.S. Cl. CPC A61M 1/107; A61M 1/1087; A61M 2205/0216; A61M 1/1006; A61M 1/1086

(57) ABSTRACT

The present invention relates generally to the field of cardiac, vascular system, and heart assistance devices. It provides the energy required to keep the blood flowing in the pulmonary and systemic circuits to a desired level, acting on one or more chambers. Actual problems of Total Artificial Heart pumping blood are design limitations, infection, hemorrhage, end organ failure, thromboembolism, device dysfunction, life span of diaphragms, and impossibility to restore the heart but with a transplant. The device is external and has four units replicating the natural heart and its dynamics, driving by a pneumatic transcutaneous system to provide the energy needed up to the desired working level of a healthy organ. Applications are on those types of surgical or clinical treatment of patients with Diastolic Heart Failure or used to treat Heart Failure with Reduced Ejection Fraction (Systolic Heart Failure), the device can be left connected permanently or for healing.

### FIELD OF THE INVENTION

The present invention relates generally to the field of cardiac, vascular system, and heart assistance devices. Applications are in those types of surgical or clinical treatment where a total or partial prosthesis is required.

### BACKGROUND OF THE INVENTION

One of the main medical problems is characterized by the inability of the left ventricle to relax properly and fill with blood. This is caused by the stiffening and impaired relaxation with normal systolic function, either due to hypertrophy or to processes such as fibrosis and infiltrative diseases. These changes cause high LV filling pressures, leading to pulmonary congestion and atrial fibrillation due to distention of the atrium. The treatment of patients with this named Diastolic Heart Failure is mainly empirical. The treatment includes modification of the underlying risk factors for the disease (such as hypertension and diabetes) and administration of medications used for treaty of Heart Failure with reduced Ejection Fraction (Systolic Heart Failure). To address this problem normal pressure in the four chambers of the heart is required and in the 1960s the first intent to build a total artificial heart (TAH) was by means of chambers where elastic rubber forced blood to flow under the impulse provided by an external fix compressor's air. In the early 1980s an electric device was borne in concept, in practice an operating one was possible in the middle 2000 at a great effort and cost, but with same performance as the previous ones, or about one-year survival time on the average, which hasn't increase since then. While considerable strides have been made in design of a TAH, two main obstacles to clinical success stem from the continued thrombogenic nature of the materials employed in the devices, coupled with design limitations. The surfaced thrombogenicity necessitates systemic antithrombotic therapy with the attendant risk of hemorrhage. Mayor remaining clinical complications with the TAH include infection, hemorrhage, end organ failure, thromboembolism and device dysfunction. Mayor causes of death with current TAH designs include sepsis, multi-organ failure, neurological death most likely due to thromboembolism, hemorrhage and problems with device fit. Thus, there is an actual need to overcome those problems by leaving the heart intact and use removable means as a partial or total artificial heart where the four chambers get a complementary help to function satisfactorily.

### SUMMARY OF THE INVENTION

Four inflatable patches to press and contract the heart wall's chambers up to the condition of normal functioning following the dynamics of those walls with proper initial set up and automation by means of a line connecting the SA (sinoatrial node) to the external hardware providing pneumatic energy as required to one or more cardiac chambers.

### BRIEF DESCRIPTION OF THE DRAWINGS

FIG. 1 is a cross section of a heart with cover and forced air lines

FIG. 2 shows the multiple layers of the surrounded cover and patches

FIG. 3 is a view of the external Control Panel showing compartments and piping circuits as guide to electronic prototype design (see FIG. 8).

FIG. 4 is a typical installation for alternative two ways of pulsed pressure and release

FIG. 5 is a cross section of FIG. 4

FIG. 6 is a graph of pressure alternative operation sequencing with over time

### DETAILED DESCRIPTION OF THE DRAWING

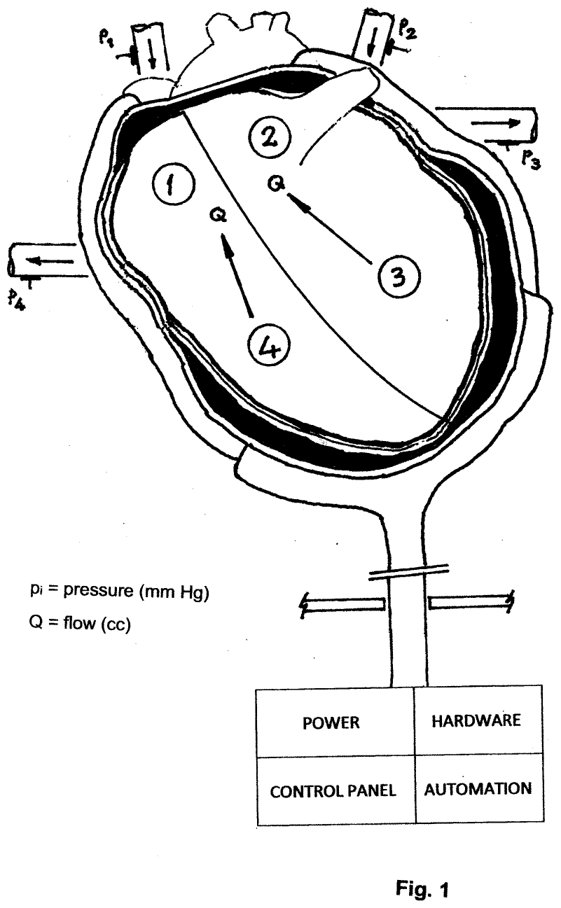

FIG. 1 In this diagram of a typical attachment numbers identifies the following parts:
1 Right Atria
2 Left Atria
3 Right Ventricle
4 Left Ventricle

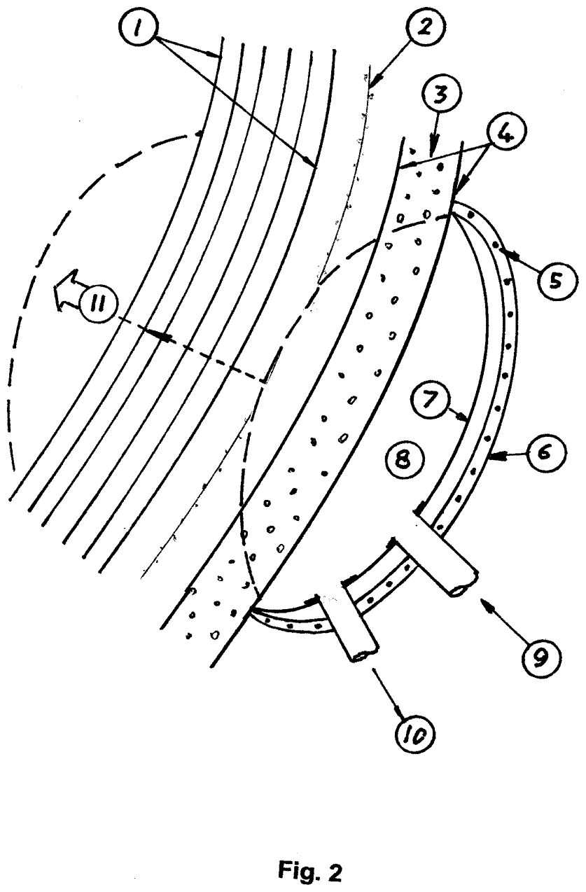

FIG. 2 In this diagram the attachment locations identify numbers the following parts:
1 Heart wall muscle
2 Flexible soft membrane lubricated both sides
3 Shock absorber layers internally sponge type
4 Elastic soft membrane
5 Mesh of embodied carbon fibers
6 Flexible reinforced external heart contouring cover
7 Elastic soft membrane
8 Working volume for compressed air
9 Air inlets
10 Air outlets
11 Expanded elastic soft membrane under pressure

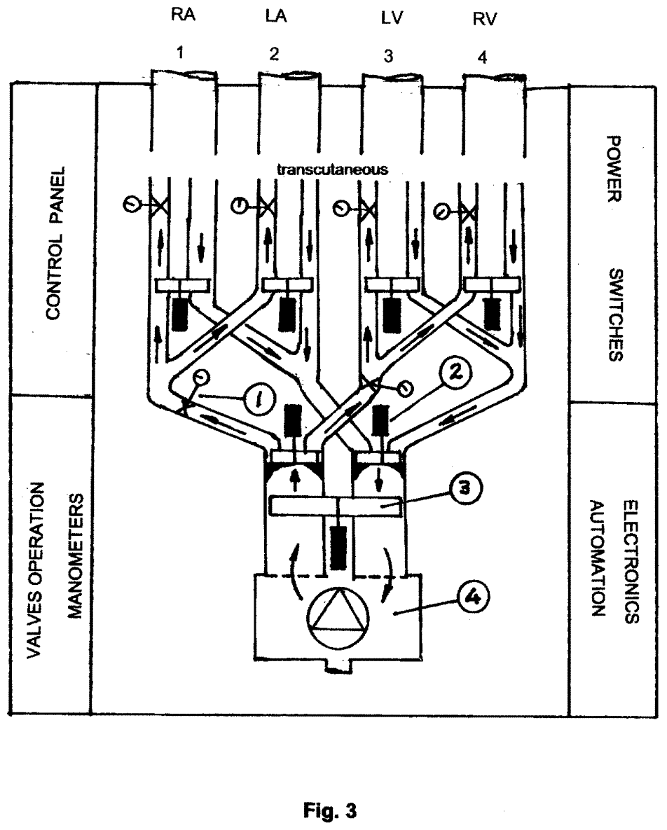

FIG. 3 In this sketch numbers identify the following parts:
1 Mini pressure gage with regulator
2 Servomotor for 180° rotation steps
3 Flat centered cylinders with opening
4 Mini air compression generator

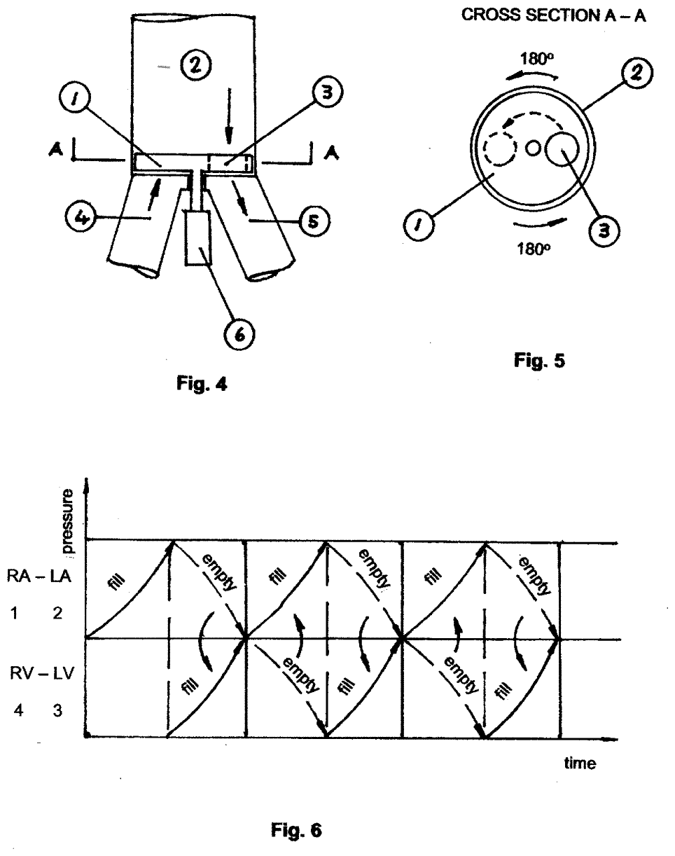

FIG. 4 Is the setup diagram for passage of air, numbers identify the following parts:
1 Centered flat cylinder
2 Main ducts containing the inlet and outlet pipes
3 Opening
4 Pressure air duct inlets
5 Release air duct outlets
6 Servo programmable motor

FIG. 5 Is a diagram of FIG. 5, numbers identify the following parts:
1 Rotating cylinder
2 Wall of duct
3 Opening

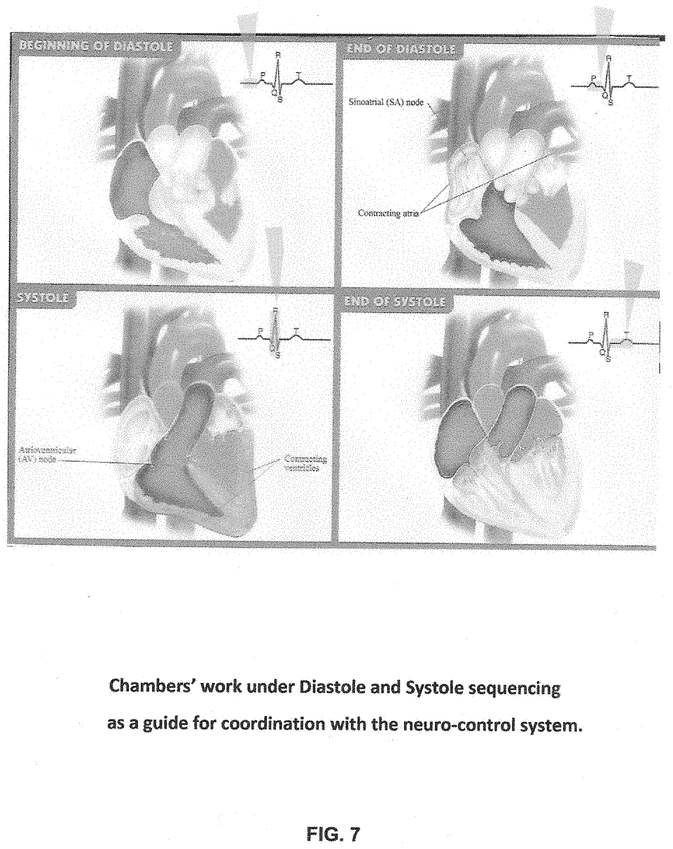

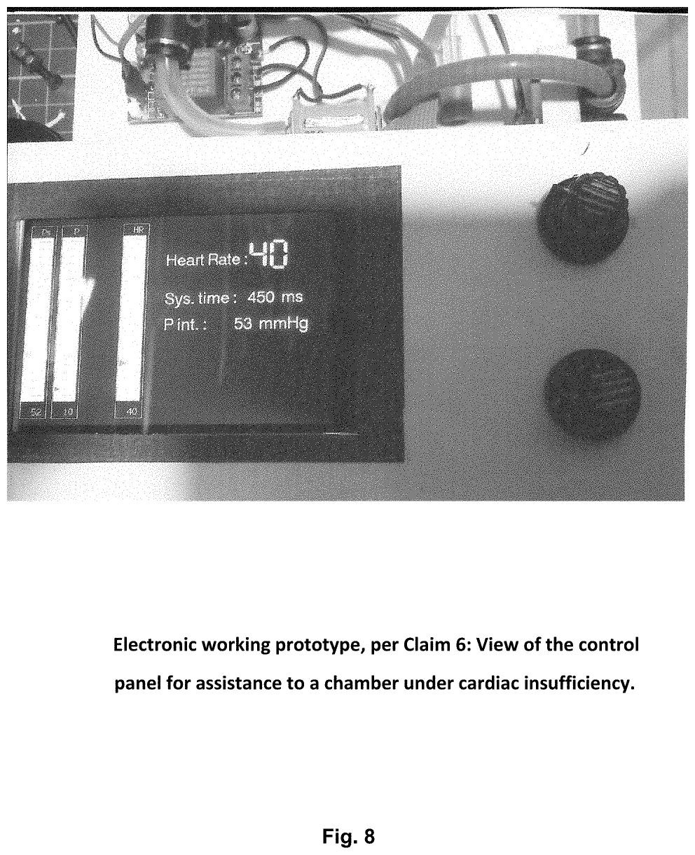

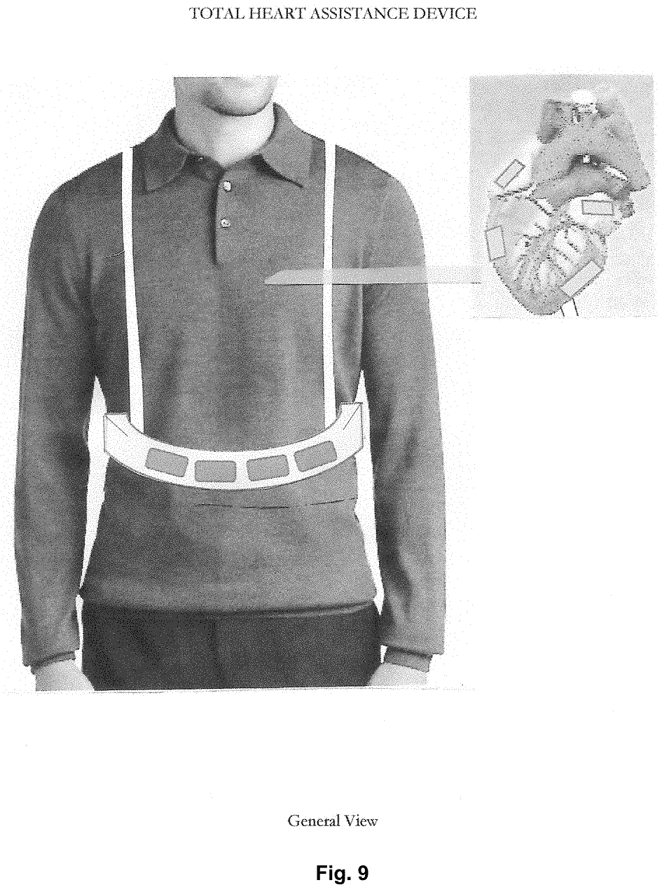

FIG. 6 shows the alternate fill and empty of chambers over time

FIG. 7 Chambers' work under Diastole and Systole sequencing. Guide for coordination with the neuro-control system.

FIG. 8 Working prototype: View of the control panel for assistance to a single chamber under cardiac insufficiency.

### DETAILED DESCRIPTION OF THE INVENTION

These heart attachments are activated by compressed air outlet of a pneumatic source, where the potential energy applied produce expansive work against the heart walls, and the consequent kinetic energy returns working as the suction force before re-entering at the inlet of the pneumatic source completing a cycle. The air flow is different for each patch to mimic or follow the total or remaining dynamic of the natural heart with which they interact fully. The compressed air fills a pocket, pouch, as a patch to add partial, or total, force such that the atria or ventricle complete its work efficiently providing blood flow, pressure and pulsation across the vascular system to reach those points in the body that require them. In doing so, strict maximum and minimum limits must be followed not to damage the heart itself or any part of the systemic or pulmonary system or irrigated tissues or organs.

FIG. 1 shows how the installation will look like before it operates. Previous work includes surgery for pass-through the skin the connecting pipes to the external pneumatic unit, control box and panel, as well as open chest to access the beating heart directly. Preparation of the patient and the attachment itself are: Use of ultrasound images for 3D dimensioning the heart at its maximum volume to produce a solid model to serve as support to put together the membranes and layers as shown in FIG. 2, where the support of the patches could be total or partial envelope of the natural heart to permit a stable addition of potential energy and action of available kinetic energy to follow the variable heart's beats. A stable system is needed because in expansion it is the ground level as the reference level for the potential energy supplied to the patches and the returning kinetic energy which contribute as suction energy for the relaxing of the heart muscle to be back at ground level. The whole attachment added power to a failing heart requires a carefully synchronization with the beating heart without stopping it, this is done by watching the ultrasound monitor dynamics, reading real time electrograph, pulse, max/min pressure before beginning to add slowly extra force and contraction/expansion up to an optimum point adjusting for optimization, using valves and servomechanisms only, always from low to high values. Operating dynamics of hardware must be at the 1/1000 sec level.

Regime is obtained by the servomotors at a 180° steps (FIG. 4 and FIG. 5), and pulse by the rotation of the one shown as number 3 in FIG. 3. This rotation mimics the natural heart 2 atria versus 2 ventricles, or any other configuration. FIG. 6 shown sequencing of filling and emptying the chambers, thou they may be partial, complementary, or total in an inactive heart when the case would be a total artificial heart (TAH) assistance device without removal and replace of the human heart.

The Control Panel can be replaced easily by a new one at any time in seconds if there is remaining activity in the patient heart. The Control Panel can operate under water as if in an accident or sport, in these cases the Panel must be waterproofed, and with a pouch of air instead of the air circuit connected to the atmosphere in the pneumatic unit.

In and out of the assistance to a beaten natural heart: The in-operation is done by looking at the pressure gages of the patient and the output from the pneumatic unit in the control external box, the reading at the patient most probably is lower than normal, then a single valve must be activated in the control box to start from zero to add pressure to the wall of the failing chamber, atria or ventricle, slowly. Continuing sequentially with the other three (if needed) chambers. The opposite will be done if the intention is an out-operation, receding to zero the reading in the control box. Once completed the failing heart will work as a failing organ, until reconnecting the control box. DANGER: If pressure is applied and the patient pressure didn't rise, is negative or is zero, means that such applied pressure work against the heart and must be stopped immediately thou slowly. When all chambers are set to work conveniently they are put fix to provide the delivered assistance. Only a trained professional is responsible to made changes thereof, by operating the governing valves, for a new assessment and set up the operation properly or disconnect the control box, if applicable.

Security and alarms for emergencies has been taken in consideration, in particular stop by run out of range of pre-stablished rates and loss of air into the thorax cavity.

While the invention has been described in conjunction with specific embodiments thereof, it is evident that many alterations, modifications, and variations will be apparent to those skilled in the art in light of the foregoing description. Accordingly, it is intended to embrace all such alterations, modifications and variations in the appended Claims, which doesn't include the patches for two reasons:
(i) This device though for final use in humans, begin as a tool to medical researchers to test parches and setting them up in place, which may deserve an additional patent.
(ii) This device works independent of a complete Total Heart Assistance Device but could be used as an implantable/external, any suggestion of a patch system cannot be used in the US or other countries that are not permitted under local regulations.
(iii) If the law permit can be used for studies in open chest animal under anesthesia and closing chest without sacrificing the animal.

1) An assembly as attachments to assist a cardiovascular biological system comprising: Four inflatable flexible patches located on the external surface of the heart chambers with an external control panel.

2) The inflatable flexible patches of claim 1, which are inflated with pressurized air conveyed from patient transcutaneous piping from a pneumatic unit on two ways: one for the pulmonary circuit and another for the systemic circuit, or four units: one for each heart's chamber independent managed.

3) The assembly attachments of claim 1, wherein the piping air flows is controlled by means of valves and servomechanisms where these activate an alternating passage of compressed air from the exhaust of compressed returning it to the pneumatic unit inlet, closing a variable-alternate cycle.

4) The assembly attachments of claim 1 where automation is managed by connecting the SA (sinoatrial node), or another point in the circuit brain-heart, to the external control panel.

5) A pneumatic unit where the functioning and piping system adapts to mimic properly the dynamic of the functioning of one or more chambers of the healthy patient's heart.

6) The piping from a pneumatic unit including alternative means, like electronic hardware including the use of proportional valves in the circuit, to provide internally compressed air from a pneumatic unit powered by transcutaneous transfer of energy.

7) Application of this assembly attachments, or parts of, to other human or animal organs to provide blood pressure, dosing drugs, lubricants, organ or tissues. healing or other needs of clinical interest.
